(19) United States
(12) Patent Application Publication
(10) Pub. No.: US 2006/0117397 A1
(43) Pub. Date: Jun. 1, 2006

(54) Imaging modalities for screening Alzheimer's disease therapeutics

(75) Inventors: Julia L. Rutkowski, Bryn Mawr, PA (US); Jack Steven Jacobsen, Bergen, NJ (US); Orest Hurko, Bryn Mawr, PA (US)

Correspondence Address: PILLSBURY WINTHROP SHAW PITTMAN, LLP, P.O. BOX 10500, MCLEAN, VA, 22102, US

(73) Assignee: Wyeth, Madison, NJ (US)

(21) Appl. No.: 11/266,230

(22) Filed: Nov. 4, 2005

Related U.S. Application Data
(60) Provisional application No. 60/625,162, filed on Nov. 5, 2004.

(51) Int. Cl. A01K 67/027

(52) U.S. Cl. 800/12; 800/18

(57) ABSTRACT

Use of animal models of neurodegenerative disorders for establishment of preclinical diagnostic and therapeutic indices, and for screening methods to identify effective preclinical therapies.

### RELATED APPLICATIONS

Priority is claimed to U.S. Provisional Patent Application No. 60/625,162, filed Nov. 5, 2004.

### FIELD OF THE INVENTION

The present invention generally relates to use of animal models of neurodegenerative disorders for identification of preclinical indices of impaired brain function and for identification of disease-modifying therapies.

### BACKGROUND OF THE INVENTION

Alzheimer's disease (AD) is the most common form of dementia, affecting about 10% of elderly people over the age of 65 years. Small et al. (1997) JAMA 278: 1363-1371. As human longevity continues to increase, AD presents a growing health crisis.

Advances in molecular neuroscience and the identification of biomarkers for neurodegenerative disease have enabled detailed descriptions of disease pathobiology. In particular, neuroimaging biomarkers have been used to describe neurodegenerative phenotypes during preclinical and early clinical disease stages. Accumulating evidence shows that neurodegenerative disorders, including Alzheimer's disease, are characterized by an extended period of progressive loss of neuronal function prior to presentation of overt clinical symptoms. Thus, intense interest is focused on the development of effective preclinical therapies that can delay or prevent clinical manifestations. See DeKosky et al. (2003) Science 302: 830-834; Silverman (2004) J. Nucl. Med. 2004 45: 594-607. Preclinical intervention to delay the onset of clinical AD by 5 years is expected to reduce prevalence of clinical AD by 50%. Additional delay could theoretically lead to virtual elimination of the disease. Brookmeyer et al. (1998) Am. J. Public Health 88: 1337-1342.

Despite enormous interest in early treatment of neurodegenerative disease, effective therapies for preclinical AD are presently unknown. To identify drugs that can retard or arrest deterioration of brain functions prior to clinical manifestation, and to assess the use of existing therapies during presymptomatic disease stages, the present invention provides relevant indices of neuroimaging biomarkers in AD animal models.

### SUMMARY OF THE INVENTION

The present invention provides methods for identification of compounds for the treatment of a neurodegenerative disorder during preclinical stages. Also provided are methods for identification of disease-modifying compounds for the treatment of a neurodegenerative disorder. The methods generally include the steps of (a) administering one or more candidate compounds to a preclinical animal model of a neurodegenerative disorder; (b) assessing changes in one or more disease biomarkers in the animal model relative to measures of the one or more disease biomarkers in a control animal; and (c) selecting a candidate compound that induces a change in one or more disease biomarkers toward measures of the one or more disease biomarkers in a control animal. As one example, the disclosed methods are useful for identifying disease-modifying drugs for the treatment of Alzheimer's disease prior to symptomatic development.

### DETAILED DESCRIPTION OF THE INVENTION

The present invention provides methods that are directed to identification of disease-modifying therapies for neurodegenerative diseases. In the case of AD, disease-modifying therapies are agents having a therapeutic effect prior to development of amyloid plaques and cognitive deterioration. The disclosed methods represent a new approach to AD drug discovery that focuses on preclinical intervention.

I. Biomarkers of Neurodegenerative Disease Diagnosis and Progression

As described herein, detection of biomarkers in animal models may be used to develop indices for preclinical diagnosis of neurodegenerative disorders and for identification of drugs having therapeutic efficacy during preclinical disease stages. In specific aspects of the invention, imaging modalities are provided for detection of impaired brain functions in AD.

The term biomarker generally refers to a characteristic, trait, or feature that can be objectively measured and evaluated as an indicator of a biological process. Biomarkers may be further described as genetic, imaging, molecular/biochemical, and clinical biomarkers. See DeKosky et al. (2003) Science 302: 830-834. These designations generally refer to methods for detection, and thus a biological condition or change may be described using one or more of the above-noted categories of biomarkers.

Disease biomarkers are biomarkers that show statistically significant variance (p<0.05) as compared to a non-disease or control condition, such as a difference of at least about 2-fold when compared with a control condition, or a difference of at least about 5-fold, or at least about 10-fold, or at least about 20-fold, or at least about 50-fold, or at least about 100-fold. Control animals used to develop imaging modalities for early disease detection are animals that do not exhibit clinical or preclinical disease measures. For assessment of disease biomarkers in an animal model comprising a transgene, induced mutation, or site-directed mutation, an animal of the parent line lacking the transgene or mutation constitutes an appropriate control animal.

The terms preclinical and presymptomatic are used interchangeably to refer to the condition of a subject prior to diagnosis of a neurodegenerative disease according to known criteria used in the art, i.e., prior to clinical disease manifestation. Preclinical patients include patients at risk for developing a neurodegenerative disease (e.g., patients carrying a genetic mutation associated with increased risk of disease), or patients presenting indices correlated with increased probability of disease development.

Clinical AD is characterized by progressive cognitive decline associated with neuronal loss, accumulation of amyloid beta (Aβ, which includes Aβ40 or Aβ42, or fragments thereof) in the neuropil (amyloid plaques) and in cerebral blood vessels (amyloid angiopathy), and the presence of neurofibrillary tangles (NFT). See Lee et al. (2001) Annu. Rev. Neurosci. 24: 1121-1159, Selkoe (2001) Physiol. Rev. 81(2):741-766. AD is further defined as dementia not otherwise diagnosed as multi-infarct dementia (MID), dementia with Lewy bodies (DLB), frontotemporal dementia (including Pick's disease), Parkinson's disease, or alcohol-related dementia (Korsakoff's syndrome).

Mutations in β-amyloid precursor protein (APP), presenilin 1 (PS1), presenilin 2 (PS2), and apoliprotein E (APOE) genes are linked to familial forms of AD. See Tandon et al. (2000) Curr. Opin. Neurol. 13: 377-383. Non-genetic risk factors also exist. For example, patients with mild cognitive impairment (MCI) show increased incidence of developing diagnosable AD. Morris et al. (2001) Arch. Neurol. 58: 397-405; Petersen (2001) Neurology 56(9):1133-1142. Additional risk factors include significant hippocampal atrophy and declining performance on recent memory testing. Petersen (2003) Mild Coqnitive Impairment: Aging to Alzheimer's Disease, Oxford University Press, New York. Preclinical AD also encompasses patients diagnosed as probable/mild AD according to National Institute of Neurological and Communicated Disorders and Stroke Criteria, which requires a demonstration of deteriorated cognition (including memory) in two or more areas of sufficient magnitude to interfere with work or social function. See McKhann et al. (1984) Neurology 34: 939-944.

The term preclinical animal model, as used herein to describe animal models for neurodegenerative disorders, refers to an animal model of a neurodegenerative disorder at a developmental stage prior to presentation of disease symptoms. Biomarkers for preclinical disease stages show comparable profiles in human patients and animal models. For example, a preclinical AD animal model is characterized by reduced cerebral blood flow and reduced glucose utilization prior to development of amyloid plaques and/or neurofibrillary tangles (NFT). Representative AD animal models are described herein below.

To identify indices for diagnosis of neurodegeneration, neuroimaging biomarkers are assessed in animal models of neurodegeneration and in human subjects at presymptomatic stages, as described in the Examples. In particular, preclinical decreases in glucose utilization, cerebral blood flow, and changes in metabolite levels may have diagnostic value when used alone or in combination with other neuroimaging biomarkers, or in combination with one or more genetic, molecular/biochemical, or clinical biomarkers. For example, additional measures of neuronal activity, neuronal integrity, neurochemistry/metabolite levels, gliosis, amyloid deposition, the presence of neurofibrillary tangles, and/or brain volume may be used to refine measures of AD-associated changes during preclinical and clinical disease progression and during post-treatment recovery.

To identify indices for therapeutic monitoring, neuroimaging biomarkers are assessed in animal models of neurodegeneration, including preclinical models, and in patients following drug administration. Indices for therapeutic monitoring are identified as measurable changes that correlate with significant changes in disease progression, including the likelihood of developing clinical stage disease.

I.A. AD Animal Models

Any relevant model for neurodegeneration may be used in the disclosed methods, including transgenic animals or animals bearing naturally occurring, induced, or targeted mutations. Several AD animal models are known in the art.

Tg2576 transgenic mice, which overexpress a mutated form of human APP695, exhibit age-dependent elevation in Aβ levels as well as neuropathological, behavioral, and metabolic impairments of AD. See Hsiao et al. (1995) Neuron 15(5):1203-1218; Hsiao et al. (1996) Science 274(5284):99-102; Hsiao (1998) Exp. Gerontol. 33(7-8):883-889; Holcomb (1999) Behav. Genet. 29(3):177-185; Niwa et al. (2002) Neurobiol. Dis. 9(1):61-68; U.S. Pat. No. 5,877,399. Cognitive defects in Tg2576 mice are detectable by about 4 months of age and prior to development of amyloid plaques. The onset of cognitive defects correlates with accumulation of brain Aβ levels. Tg2576 mice having elevated levels of Aβ also show impaired production of vascular relaxing factors by cerebral endothelial cells, an inability to maintain adequate blood flow during hypotension, disruption of activity-based enhancement of cerebral blood flow, and reduction of cerebral glucose utilization (Niwa et al. (2002) Neurobiol. Dis. 9(1):61-68; Niwa et al. (2002) Am. J. Physiol. Heart Circ. Physiol. 283(1):H315-23; Iadecola et al. (1999) Acta. Neuropathol. (Berl.) 98: 9-14). PSAPP mice overexpress both mutant amyloid precursor protein and mutant presenilin 1 transgenes and show rapid disease progression. Aβ deposition is detectable in cingulate cortex by about 10 weeks of age. By 6 months, amyloid deposition is widespread, including deposition in the hippocampus, cortex, and other brain regions. See McGowan et al. (1999) Neurobiol. Dis. 6(4):231-244.

Additional AD animal models that may be used in the disclosed methods include an animal having a transgene that encodes APP and at least one mutation associated with Alzheimer's disease, for example, the Swedish mutation (lysine⁵⁹⁵-methionine⁵⁹⁶ mutated to asparagine⁵⁹⁵-leucine⁵⁹⁶) (U.S. Pat. Nos. 6,509,515 and 6,586,656); PDAPP transgenic mice, which overexpress a minigene containing human APPV717F mutation (Games et al. (1995) Nature 373: 523-527; U.S. Pat. No. 6,717,031); an animal model having a transgene that encodes a 99 to 103 amino acid carboxy-terminus portion of human APP (U.S. Pat. No. 6,037,521); an animal model having a transgene encoding the carboxyl-terminal 100 amino acids of human APP (U.S. Pat. Nos. 5,849,999 and 5,894,078); an animal model having a transgene encoding human APP751 and APP695 (U.S. Pat. No. 5,850,003); an animal model having a transgene encoding a mutant protein product of a mutated FAD presenilin-1 (PS-1) gene and human APP695 Swedish mutation (U.S. Pat. No. 5,898,094); an animal model having a gene-targeted mutated FAD presenilin-1 (PS-1) gene and a human APP695 Swedish mutation (U.S. Pat. No. 6,734,336); an animal model having a gene-targeted mutated FAD presenilin-1 (PS-1) gene, a human FAD Swedish mutation, and a humanized Aβ mutation (U.S. Pat. No. 6,734,336); an animal model designated TgCRND8 having a transgene encoding a human APP695 mutation, which further includes K670N, M671 L, and V717F mutations (U.S. Patent Publication No. 0030093822); an animal model having a transgene encoding APP770 with a mutation at position 717 (U.S. Pat. No. 6,300,540); an animal model having a transgene encoding tau protein (U.S. Pat. Nos. 6,593,512 and 6,664,443); an animal model having a transgene encoding human receptor for advanced glycation endproducts (RAGE) and also encoding human APP bearing mutations linked to familial Alzheimer's disease (U.S. Pat. No. 6,563,015); a transgenic animal model that overexpresses TGF-β1, optionally in combination with expression of human APP (U.S. Pat. No. 6,175,057); and animals prepared based on combinations of one or more of the foregoing mutations.

An animal model is typically a rodent animal model, however, models prepared in other relevant animals (e.g., primate, cat, rabbit, guinea pig, goat, horse, cow, etc.) are also useful in the invention.

I.B. Neuroimaging Biomarkers

The term neuroimaging biomarker is used herein to refer to changes in functional brain activity, or other neural changes, that are detectable in vivo. Neuroimaging biomarkers may be assessed in a subject using imaging methods such as magnetic source imaging and scintigraphic techniques.

For AD patients, including patients diagnosed as probable AD and patients at risk of developing AD, relevant markers of functional brain activities include cerebral atrophy/brain volume (e.g., assessed by MRI), cerebral blood flow (CBF) or cerebral blood volume (CBV) (e.g., assessed by MRI), oxygen uptake (e.g., assessed by ¹⁵O₂ SPECT), glucose uptake (e.g., assessed by [¹⁸F]flurodeoxyglucose (FDG) PET), levels of brain metabolites, such as N-acetylaspartate and myo-inositol (e.g., assessed by MRS), microglia activation (e.g., assessed by PK11195 (1-(2-chlorophenyl)-N-methyl-N-(1-methyl-propyl)-3-isoquinolin carboxamide) PET), and amyloid plaque deposition (e.g., assessed by ¹¹C-BIP (¹¹C-Pittsburgh Compound B) PET). While amyloid plaques and neurofibrillary tangles are indicators of clinical AD, abnormalities in other neuroimaging biomarkers may be detected during preclinical stages. See e.g., Schott (2002) Proc. Natl. Acad. Sci. U.S.A. 99(7):47034707 (elevated atrophy rate of medial temporal lobe structures detected at early stages of AD, and extrapolation of atrophy rates suggests that pathological atrophy occurs several years prior to symptomatic disease); Rusinek et al. Radiology 229: 691-696 (longitudinal study of patients over 6 years shows that an increased rate of atrophy of medial temporal lobe predicts future cognitive decline); Bookheimer et al. (2000) N. Engl. J. Med. 343(7):450456 and Smith et al. (1999) Neurology 53: 1391-1396 (cerebrovascular changes are also observed in patients with AD-linked genetic mutations prior to development of symptomatic disease); Reiman et al. (2001) Proc. Natl. Acad. Sci. U.S.A. 98(6):3334-3339; Small et al. (2000) Proc. Natl. Acad. Sci. U.S.A. 97(11):6037-6042 (altered glucose utilization in AD patients at early disease stages); Cagnin et al., (2002) Eur. Neuropsychopharmacol. 12: 581-586 (patients with minimal cognitive impairment show neuroinflammation in areas that subsequently undergo atrophy prior to development of clinical dementia); Jessen et al. (2001) Neurology 57: 930-932 and Jaarsma et al. (1994) J. Neurol. Sci. 127: 230-233 (reduced levels of N-acetylaspartate in AD patients); and Parnetti et al. (1996) J. Am. Geriat. Soc. 44: 133-138 (elevated levels of myo-inositol in AD patients).

I.B.1. Magnetic Source Imaging

Neuroimaging of blood flow and brain volume may be performed using magnetic resonance imaging (MRI), which creates images based on the relative relaxation rates of water protons in unique chemical environments. As used herein, the term magnetic resonance imaging or MRI refers to magnetic source techniques alternatively described as one or more of conventional magnetic resonance imaging, magnetization transfer imaging (MTI), proton magnetic resonance spectroscopy (MRS), diffusion-weighted imaging (DWI), perfusion-based imaging, and functional MR imaging (fMRI). See e.g., Rovaris et al. (2001) J. Neurol. Sci. 186 Suppl 1:S3-9; Pomper & Port (2000) Magn. Reson. Imaging Clin. N. Am. 8:691-713; and references cited therein.

ASL (arterial spin labeling) and CASL (continuous arterial spin labeling) are functional magnetic resonance imaging techniques that depend on changes in inflowing blood spins that are in a different magnetic state than that of the static tissue. MR images are sensitized by magnetically labeling blood flowing into a tissue slice of interest. This perfusion measurement is completely non-invasive and does not require administration of contrast agents. Perfusion-weighted images are generated by the subtraction of an image obtained from tissue following inflowing spins from an image in which spin labeling is not performed. Perfusion changes may be quantified by comparison to other parameters, for example, tissue T1 and the efficiency of spin labeling. CASL involves administration of a series of radiofrequency pulses, whereby blood water is repeatedly saturated. The exchange of labeled spins and brain tissue water approaches a steady state, such that the regional magnetization in the brain is directly related to cerebral blood flow. See e.g., Calamante et al. (1999) J. Cereb. Blood Flow & Metab. 19: 701-735; Detre et al. (1992) Magn. Reson. Med. 23(1): 37-45; and Floyd et al. (2003) J. Magn. Reson. Imaging 18(6): 649-655.

For MRI techniques other than ASL/CASL, a contrast agent may be used to facilitate signal detection. Contrast agents for magnetic source imaging include but are not limited to paramagnetic or superparamagnetic ions, iron oxide particles, for example monocrystalline iron oxide nanoparticle (MION) (Weissleder et al. (1992) Radiology 182(2):381-385.; Shen (1993) Magn. Reson. Med. 29(5): 599-604) and water soluble contrast agents. Paramagnetic and superparamagnetic ions may be selected from the group of metals including iron, copper, manganese, chromium, erbium, europium, dysprosium, holmium and gadolinium. Images derived used a magnetic source may be acquired using, for example, a superconducting quantum interference device magnetometer (SQUID, available with instruction from Quantum Design of San Diego, Calif.). See U.S. Pat. No. 5,738,837.

Representative methods for ASL detection of cerebral blood flow in AD animal models and in AD patients are described in Examples 3 and 7, respectively. Additional representative methods for magnetic resonance imaging of blood flow in animal models may be found in van Bruggen et al. (1998) J. Cereb. Blood Flow Metab. 18(11):1178-1183; Mandeville et al. (1998) Magn. Reson. Med. 39: 615-624; Mueggler et al. (2001) Magn. Reson. Med. 46: 292-298.

Imaging of regional brain metabolism may be performed using MRS, which measures cellular activity on the basis of the levels of phospholipids, hi-energy compounds, inorganic phosphates, neurotransmitters, and amino acids. For example, energy metabolism in brain may be assessed by determining levels of adenylate and creatine phosphates, (ATP, ADP, AMP, CP), intermediates of glycolysis and the tricarboxylic acid (TCA) cycle, TCA enzymes, oxidative phosphorylation, electron transfer chain complexes, and ATPases (e.g., K⁺-ATPase, Ca²⁺-ATPase).

Representative methods for assessing neurochemical profiles in AD animal models and in AD patients using MRS are described in Examples 4 and 8, respectively. Additional methods for determination of metabolite levels in animal models and patients are described in Dedeoglu et al. (2004) Brain Res. 1012: 60-65 and in Sanacora et al. (2002) Am. J. Psychiatry 159: 663-665.

I.B.2. Scintigraphic Imaging

Scintigraphic imaging generally refers to radiolabel-based imaging and includes positron emission tomography (PET), single photon emission computed tomography (SPECT), gamma camera imaging, and rectilinear scanning. Most SPECT systems are based on the use of one or more gamma cameras that are rotated about the subject of analysis, and thus integrate radioactivity in more than one dimension. PET systems comprise an array of detectors in a ring that also detect radioactivity in multiple dimensions. A gamma camera and a rectilinear scanner each represent instruments that detect radioactivity in a single plane. Related devices for scintigraphic imaging may also be used, such as a radio-imaging device that includes a plurality of sensors with collimating structures having a common source focus.

Scintigraphic techniques may be used for neuroimaging of AD biomarkers, including oxygen and glucose utilization, microglial activation, and late-stage markers, such as amyloid plaques and neurofibrillary tangles (NFT). Changes in glucose utilization are detected in AD animal models and in AD patients prior to accumulation of Aβ and neurofibrillary tangles. Therefore, these measures are particularly useful for developing diagnostic and therapeutic indices during preclinical stages. See Niwa et al. (2002) Neurobiol. Disease 9: 61-68 and Alsop et al. (2000) Ann. Neurol. 47: 93-100. Other ligands may be used for scintigraphic imaging, i.e., any ligand that specifically bind to molecules involved in functional brain activity (e.g., receptors, antibodies, enzymes, and ion channels), that can be delivered to the brain in amounts sufficient for imaging, and that is rapidly cleared from normal brain tissue.

Detectable labels for scintigraphic imaging include ⁵⁷cobalt, ⁶²copper, ⁶⁴copper, ⁶⁷gallium ⁵¹chromium, ¹⁶⁶holmium, ¹¹¹indium, ^{113m}indium, ¹²²iodine, ¹²³iodine, ¹²⁵iodine, ¹³¹iodine, ¹³²iodine, krypton, ¹⁷⁷lutetium, ¹⁹⁷mercury, ²⁰³mercury, ⁹⁹ molybdenum, ⁴²potassium, ³²phosphorous, ¹⁸⁶rhenium, ⁸¹rubidium, ⁸²rubidium, ⁷²selenium, ⁷⁵selenium, ^{99m}technetium, ²⁰¹thallium, ¹²⁷xenon, ¹³³xenon, ¹⁶⁹ytterbium, and ⁶²zinc. Cyclotron radioisotopes may also be used, for example, ¹¹carbon, ¹³nitrogen, ¹⁵oxygen, and ¹⁸fluorine.

Representative methods for neuroimaging of glucose utilization by [¹⁸F]fluordeoxyglucose (FDG) PET in AD animal models and AD patients are described in Examples 2 and 6, respectively. Neuroimaging of activated microglia may be performed as described by Shah et al. (1994) Nucl. Med. Biol. 21: 573-581, or by Ramsay et al. (1992) Lancet 339(8800): 1054-1055. PK11195 [1-(2-chlorophenyl)-N-methyl-N-(1-methyl-propyl)-3-isoquinolin carboxamide), which is a ligand for the peripheral benzodiazepine binding site abundant on phagocytic cells, is used as a tracer molecule. Amyloid plaques may be imaged using benzothiazole amyloid-binding tracer molecules such as PIB or BTA (2-(4′ methylaminophenyl)benzothiazole). Additional tracer molecules for detection of amyloid deposits are described in Mathis et al. (2002) Bioorg. Med. Chem. Lett. 12: 295-298; Lee et al. (2003) Nucl. Med. Biol. 30(6):573-580; Wang et al. (2002) J. Mol. Neurosci. 19(1-2):11-16. AD patients show retention of the PIB amyloid tracer in areas of association cortex known to contain substantial amyloid deposits, including frontal cortex, parietal, temporal, and occipital cortex, and striatum. Klunk et al. (2004) Ann. Neurol. 55: 306-319. Neurofibrillary tangles may be visualized using a ligand such as 1,1-dicyano-2-[6-(dimethylamino)napthalen-2-y]propene (FDDNP) (Agdeppa et al. (2003) Mol. Imaging Biol. 5(6): 404-17). See also Small et al. (2002) J. Mol. Neurosci. 19: 323-327 and U.S. Pat. No. 6,660,530.

I.C. Molecular Biomarkers

The terms biochemical biomarker or molecular biomarker generally refer to molecules that are detectable in a biological sample such as CSF or serum using an in vitro assay. Molecular biomarkers are readily detected using routine techniques such as ELISA (enzyme-linked immunosorbent assay), Western blot, analysis of stained histologic tissues, etc. In general, such assays use an antibody or antibody fragment that specifically binds the molecular biomarker and which includes a detectable label (e.g., a radiolabel enzyme, fluorophore, etc.). Molecular biomarkers may include in vitro detection of the above-noted neuroimaging biomarkers, e.g. detection of elevated Aβ using an in vitro assay.

Additional molecular biomarkers that may be detected in AD patients include isoprostanes, phosphorylated Tau (pTau) and total CSF Tau (tTau) (Hampel et al. (2004) Dement. Geriatr. Cogn. Disord. 17: 350-354; Hampel et al. (2004) J. Neural Transm. 111(3): 247-272), and neuronal thread protein (NTP) (de la Monte et al. (2002) Front Biosci. 7: d989-996). Additional molecular biomarkers are described in U.S. Pat. No. 6,717,031.

I.D. Clinical Biomarkers

The term clinical biomarker generally refers to a quantifiable measure of clinically manifested neurodegenerative disease. Useful clinical biomarkers for assessing disease in both AD animal models and in AD patients include contextual fear conditioning (Corcoran et al. (2002) Leam. Mem. 9(5): 243-252.) and conditioned eye blink tests. Additional representative clinical biomarkers for use in AD patients include CANTAB (Cambridge Neuropsychological Test Automated Battery) Paired Associates Learning test (Blackwell et al. (2004) Dement. Geriatr. Cogn. Disord. 2004; 17(1-2):42-48.), the Cognitive Log (Alderson et al. (2003) Arch. Phys. Med. Rehabil. 84: 668-672), the Mini-Cog (Borson 2003), mini-mental state examination (MMSE) (Folstein et al. (1975) J. Psychiatr. Res. 12: 189-198), the Alzheimer's disease assessment scale-cognitive (ADAS-Cog) (Rosen et al. (1984) Am. J. Psychiatry 141: 1356-1364), and the computerized neuropsychological test battery (CNTB) (Caramelli et al. (2004) Arq. Neuropsiquiatr. 62(2B): 379-384).

II. Screening Methods

The present invention provides methods for identification of disease-modifying therapies, i.e., therapies that exert an effect on the pathoetiological mechanisms of disease, by assaying preclinical indices of disease. Detection of biomarkers in preclinical animal models is also useful for characterizing existing symptomatic treatments, i.e., drugs directed to amelioration of primary or secondary disease symptoms rather than to treatment of the underlying pathophysiology, which may reveal previously unrecognized disease-modifying effects. While some preclinical indices of neurodegeneration are known, drugs for administration to patients at preclinical stages to avert progression to clinical stages, and methods for identifying drugs active at preclinical stages, have not been described. Thus, the present invention further provides novel disease-modifying drugs identified by performance of the disclosed screening assays.

According to the disclosed methods, screening assays are performed in preclinical animal models by assaying changes in disease biomarkers. Efficacy of identified therapies is confirmed in human patients by assessment of the same biomarkers. See Examples 1-10. As described herein above, the neuroimaging modalities are particularly useful for characterization of these early changes.

For example, a screening assay of the invention may comprise (a) administering one or more candidate compounds to an animal model of preclinical stages of a neurodegenerative disorder; (b) assessing changes in one or more neuroimaging biomarkers in the animal model relative to measures of the one or more neuroimaging biomarkers in a control animal; and (c) selecting a candidate compound that induces a change in one or more neuroimaging biomarkers toward measures of the one or more neuroimaging biomarkers in a control animal. See Examples 1-4.

Criteria for identification of drug efficacy include measurement of at least one disease biomarker, e.g., a neuroimaging biomarker, and preferably a combination of two or more neuroimaging biomarkers. Drug efficacy may be further defined by additional assessment of genetic biomarkers, biochemical/molecular biomarkers, and clinical biomarkers.

Control animals used in screening assays are animals that receive a placebo in lieu of treatment. Compounds that promote restoration of neurodegenerative disease biomarkers toward control values are referred to herein as neuroprotective agents. Preferably, a neuroprotective agent shows statistically significant variance (p<0.05) as compared to the pretreatment condition or as compared to a control animal that received a placebo treatment. For example, a neuroprotective agent is identified wherein biomarkers are changed at least about 2-fold when compared with a control animal, or a difference of at least 5-fold, or at least about 10-fold, or at least about 20-fold, or at least about 50-fold, or at least about 100-fold. The present invention further provides neuroprotective agents identified by the disclosed screening methods.

The term drug as used herein refers to any substance having biological activity, including any natural or synthetic chemical molecule, including small molecules (e.g., organic compounds), peptides, proteins, sugars, lipids, fatty acids, steroids, purines, pyrimidines, or nucleic acids. Relevant candidate drugs include drugs capable of modifying cognition, for example, drugs that modulate neurotransmitter levels (e.g., acetylcholinesterase inhibitors, cholinergic receptor agonists or serotonin receptor antagonists), drugs that modulate the level of soluble Aβ or amyloid plaque burden (e.g., γ-secretase inhibitors, β-secretase inhibitors, antibody therapies, and degradative enzymes), and drugs that protect neuronal integrity (e.g., antioxidants, kinase inhibitors, caspase inhibitors, and hormones). Other representative candidate drugs for use in the disclosed screening methods include cholinesterase inhibitors, (e.g., tacrine (COGNEX®), donepezil hydrocholoride (ARICEPT®), rivastigmine (EXELON®) and galantamine (REMINYL®)), cysteinyl protease inhibitors, galantamine, an inhibitor of receptor for advanced glycation endproduct (RAGE) 5-HTR1a antagonists, 5-HT6 antagonists, BACE inhibitors, α-secretase, immunophilins, caspase-3 inhibitors, Src kinase inhibitors, PDE4 inhibitors, TPA activators, AMPA modulators, M4 agonists, JNK3 inhibitors, LXR agonists, H3 antagonists, angiotensin IV antagonists, etc.

III. Clinical Applications

The indices of AD progression, which are identified in AD animal models and in AD patients using neuroimaging modalities as disclosed herein, may be used for detection of impaired brain functions prior to clinical disease manifestation. These indices are also useful for monitoring improvement of brain functions in response to preclinical therapy.

Drugs for preclinical therapy, which are identified in screening assays that employ animal models of preclinical neurodegenerative disease, are intended for use in human patients. Efficacy and therapeutic response of drugs that are effective in animal models may be similarly assessed using neuroimaging biomarkers in human patients. See Example 2. Disease-modifying drugs identified in the disclosed screening assays may be used for therapy of patients at preclinical stages as well as clinical stages.

Drugs for preclinical therapy of AD may also be used for treatment of related amyloidogenic diseases, such as scrapie, transmissible spongioform encephalopathies (TSEs), hereditary cerebral hemorrhage with amyloidosis Icelandic-type (HCHWA-1), hereditary cerebral hemorrhage with amyloidosis Dutch-type (HCHWA-D), familial Mediterranean fever, familial amyloid nephropathy with urticaria and deafness (Muckle-Wells syndrome), myeloma or macroglobulinernia-associated idopathy associated with amyloid, familial amyloid polyneuropathy (Portuguese), familial amyloid cardiomyopathy (Danish), systemic senile amyloidosis, familial amyloid polyneuropathy (Iowa), familial amyloidosis (Finnish), Gerstmann-Staussler-Scheinker syndrome, medullary carcinoma of thyroid, isolated atrial amyloid, diabetes type II, and insulinoma.

Neuroprotective agents identified as described herein may be formulated for safe and efficacious clinical use. Suitable formulations for administration to a subject include aqueous and non-aqueous sterile injection solutions which may contain anti-oxidants, buffers, bacteriostats, antibacterial and antifungal agents (e.g., parabens, chlorobutanol, phenol, ascorbic acid, and thimerosal), solutes that render the formulation isotonic with the bodily fluids of the intended recipient (e.g., sugars, salts, and polyalcohols), suspending agents and thickening agents. Suitable solvents include water, ethanol, polyol (e.g., glycerol, propylene glycol, and liquid polyethylene glycol), and mixtures thereof. The formulations may be presented in unit-dose or multi-dose containers, for example, sealed ampoules and vials, and may be stored in a frozen or freeze-dried (lyophilized) condition requiring only the addition of sterile liquid carrier immediately prior to use for administration to a subject.

Neuroprotective agents may also be formulated to include a pharmaceutically acceptable carrier, for example, large slowly metabolized macromolecules such as proteins, polypeptides, liposomes, polysaccharides, polylactic acids, polyglycolic acids, polymeric amino acids, amino acid copolymers and inactive virus particles. Pharmaceutically acceptable salts may also be used, for example mineral acid salts, such as hydrochlorides, hydrobromides, phosphates and sulfates, or salts of organic acids, such as acetates, propionates, malonates and benzoates. Formulations may additionally contain liquids such as water, saline, glycerol, and ethanol, and/or auxiliary substances, such as wetting or emulsifying agents or pH buffering substances, may be present in such compositions.

The present invention provides methods for treatment of neurodegenerative disorders during preclinical and/or clinical stages via administration of an effective amount of a neuroprotective agent. The term effective dose is used herein to describe an amount of a neuroprotective agent sufficient to elicit a desired biological response. For example, when administered to an AD animal model or to an AD patient, an effective dose comprises an amount sufficient to retard or arrest neurodegeneration or brain atrophy, or an amount sufficient to promote brain function, for example, an amount sufficient to increase cerebral blood flow and/or glucose utilization.

Neuroprotective agents of the invention are formulated and administered to achieve effective doses in CSF and brain. For example, neuroprotective agents having lipophilic properties that enable crossing of the brain-blood barrier may be administered intravenously. Administration may also comprise intrathecal, intralesional, intraperitoneal, or intramuscular injection; infusion; bombardment; topical, nasal, oral, ocular, or otic delivery. These administration routes may also be employed for delivery of candidate compounds in screening assays.

A therapeutically effective dose and administration regimen may be estimated in animal models, such as those described herein. Such information may then be used to determine useful doses and routes for administration in humans. Typically, a minimal dose is administered, and the dose is escalated in the absence of dose-limiting cytotoxicity. Determination and adjustment of an effective dose, as well as evaluation of when and how to make such adjustments, are known to those of ordinary skill in the art of medicine. The selected dosage level and regimen will depend upon a variety of factors including the activity and stability (i.e., half life) of the therapeutic composition, formulation, the route of administration, combination with other drugs or treatments, the severity of the disease or disorder to be detected and/or treated, and the physical condition and prior medical history of the subject being treated.

Neuroprotective agents of the invention may be used in combination with other therapies, for example, known symptomatic therapies for neurodegenerative disorders; anti-inflammatory agents; immunogens, e.g., amyloid-β or non-amyloidogenic peptides for immunization therapies (Schenk et al. (1999) Nature 400(6740): 173-177; U.S. Pat. No. 6,713,450); and/or agents for clearance of amyloid plaques, such as anti-amyloid-p antibodies (Bacskai et al. (2001) Nature Med. 7(3): 369-372; Bard et al. (2000) Nature Med. 6(8): 916-919) or Cimicifuga extracts (U.S. Pat. No. 6,649,196).

For administration of multiple therapeutic agents, including clinical administration or use in screening assays, neuroprotective agents and additional therapeutic agents are administered within any time frame suitable for performance of the intended therapy or diagnosis. Thus, the single agents may be administered substantially simultaneously (i.e., as a single formulation or within minutes or hours) or consecutively in any order. For example, single agent treatments may be administered within about 1 year of each other, such as within about 10, 8, 6, 4, or 2 months, or within 4, 3, 2 or 1 week(s), or within about 5, 4, 3, 2 or 1 day(s).

For additional guidance regarding formulation, dose, administration regimen, and measurable therapeutic outcomes, see Berkow et al. (2000) The Merck Manual of Medical Information, Merck & Co., Inc., Whitehouse Station, New Jersey; Ebadi (1998) CRC Desk Reference of Clinical Pharmacology, CRC Press, Boca Raton, Fla.; Gennaro (2000) Remington: The Science and Practice of Pharmacy, Lippincott, Williams & Wilkins, Philadelphia, Pa.; Katzung (2001) Basic & Clinical Pharmacology, Lange Medical Books/McGraw-Hill Medical Pub. Div., New York; Hardman et al. (2001) Goodman & Gilman's the Pharmacological Basis of Therapeutics, The McGraw-Hill Companies, Columbus, Ohio; Speight & Holford (1997) Avery's Drug Treatment: A Guide to the Properties, Choices, Therapeutic Use and Economic Value of Drugs in Disease Management, Lippincott, Williams, & Wilkins, Philadelphia, Pa.

### EXAMPLES

The following examples have been included to illustrate modes of the invention. Certain aspects of the following examples are described in terms of techniques and procedures found or contemplated by the present co-inventors to work well in the practice of the invention. In view of the present disclosure and the general level of skill in the art, those of skill will appreciate that the following examples are intended to be exemplary only and that numerous changes, modifications, and alterations may be employed without departing from the scope of the invention.

### Example 1

### Assessment of Preclinical Biomarkers in Animal Models of Alzheimer's Disease

Neuroimaging biomarkers are assessed in AD animal models and appropriate control animals. Representative AD animal models include Tg2576 transgenic mice and PSAAP mice, which develop more severe plaque pathology at an earlier stage.

Tg2576 or PSAPP transgenic mice are compared to wild type littermates to determine whether alterations in disease biomarkers are detectable prior to amyloid deposition. Four groups of Tg2576 transgenic and wild type mice, each group having 4-10 littermates, are analyzed at 5 months and at 16 months of age. In view of their more rapid disease progression, PSAPP animals are tested at 2 months and 6 months of age. An additional observational study is performed wherein Tg2576 and PSAPP mice are evaluated monthly beginning at 4 months and 2 months of age, respectively. For each test group, disease biomarkers are assessed at the same time on 3 consecutive days. The variability and dynamic range of each biomarker is determined among animals and among multiple assessments in a single animal. Affected brain regions are identified and used to establish indices for AD diagnosis, including presymptomatic diagnosis. For example, prior to observation of amyloid deposition, a reduced level of glucose utilization and cerebral blood flow is observed in AD animal models as compared to control animals.

To identify indices for therapeutic monitoring, including at preclinical stages, disease biomarkers are assessed in AD animal models following drug administration, including drugs identified as described in Example 10 and known drugs currently used for symptomatic therapy. For example, a short-term treatment study is conducted in which Tg2576 or PSAPP mice are treated for 1 week, beginning when initial observations of significant changes in hippocampal function are detected in the above-noted observational study. Tg2576 mice are administered escalating drug doses followed by weekly monitoring of disease biomarkers until their return to control levels (i.e., relevant measures of biomarkers in control animals). For evaluation of known symptomatic drugs at preclinical stages, the drug is administered in a therapeutically effective amount, e.g., an amount effective to increase cognitive performance. A long-term treatment study is also conducted in which Tg2576 or PSMP mice are treated for 3 months and compared to untreated control mice at monthly intervals until the earlier of 6 months or until disease biomarkers return to control levels.

### Example 2

### Determination of Glucose Utilization in Animal Models of Alzheimer's Disease

Glucose utilization is assessed in AD animal models, as described in Example 1, using [¹⁸F]flurodeoxyglucose (FDG) PET, essentially as described by Toyama et al. (2004) J. Nucl. Med. 45(8): 1398-1405. FDG is administered to AD animal models and control animals under normoglycemic conditions (e.g., under isoflurane anesthesia or in an awake state). Regional glucose utilization is determined using a small animal PET scanner.

### Example 3

### Determination of Cerebral Blood Flow in Animal Models of Alzheimer's Disease

Cerebral blood flow is measured in AD animal models (see Example 1) using ASL, essentially as described by Detre et al. (1992) Magn. Reson. Med. 23(1):37-45. Blood water flowing to the brain is saturated in the neck region using a slice-selective saturation imaging sequence. Proton MRI is carried out on a 4.7 T NMR spectrometer with a 40 cm magnet bore equipped with a 15 cm diameter gradient insert. Representative conditions using a 7 cm diameter volume coil are as follows: recovery time=2 seconds, echo time=30 ms, field of view=5 cm, slice thickness=2 mm, matrix size=64×64. Gradients are applied in the imaging sequence so as to eliminate the contribution of flowing spins in blood vessels while minimally perturbing tissue water. Inversion of the inflowing spins is accomplished by continuously applying a low-power radiofrequency field in the presence of a magnetic field gradient during the TR period. Following exchange of saturated spins with bulk water in the brain, the regional concentration of saturated spins is determined by the regional blood flow and regional T1. Distal saturation applied equidistantly outside the brain serves as a control for effects of the saturation pulses. See also Williams et al. (1992) Proc. Natl. Acad. Sci. U.S.A. 89: 212-216 and Sun et al. (2004) Magn. Reson. Med. 51:893-899.

A multi-slice MRI sequence may be obtained by using a multiple-coil system, which may reduce saturation of macromolecular spins during arterial spin labeling. For example, a two-coil system may be used, which consists of one small surface coil for labeling the arterial water spins and a decoupled head coil for MRI. See Silva et al. (1995) Magn. Reson. Med. 33(2): 209-214.

### Example 4

### Determination of Neurochemical Profile in Animal Models of Alzheimer's Disease

Neurochemical profile is assessed in AD animal models (see Example 1) using MR imaging in conjunction with spectroscopy at 4.7 T using a 20 mm sinusoidal birdcage coil. T2-weighted spin echo MR images are collected with a spatial resolution of about 0.1×0.2 mm in plane and 1 mm slices. Voxels are centered over selected brain regions, and voxel sizes are adjusted according to brain size. Spectra are recorded, for example, using a PRESS technique with a TR of 2.2 seconds and TE values of 144 and 272 milliseconds. Data are processed using curve fitting of the spectra and intensities are integrated. Resonance integrals are normalized to the creatine peak. Multiple TE values are collected for correction of T2 data between AD animal models and control animals. Changes in metabolites are determined, including for example, one or more of N-acetylaspartate, myo-inositol, taurine, scyllo-inositol, choline, Cr-CH3, GSH, aspartate, glutamine, succinate, glutamate, GABA, alanine, and lactate.

### Example 5

### Assessment of Preclinical Biomarkers in AD Patients

Double blind cross-over studies are conducted involving patients having probable/mild AD selected according to the DSM-IV-TR and National Institute of Neurological and Communicated Disorders and Stroke Criteria (McKhann et al. (1984) Neurology 34: 939-944). In a short-term study, patients are treated and assessed at 2-week intervals over a period of two months. For long-term evaluation, patients are treated for six months with bimonthly monitoring. Studies are also conducted involving patients having elevated risk of developing AD.

### Example 6

### Determination of Glucose Utilization in AD Patients

Glucose uptake is assessed in patients having probable/mild AD or in patients having an elevated risk of developing AD (see Example 5) using [¹⁸F]flurodeoxyglucose (FDG) PET, essentially as described by Small et al. (2000) Proc. Natl. Acad. Sci. U.S.A. 97(11):6037-6042. Tracer injections of 370 MBq FDG are administered via intravenous injection. Scans are performed about 40 minutes after FDG injection using, for example, a CTI/Seimens 831-08 or CTI 962 scanner. Scans are acquired parallel to the canthomeatal line, and a transmission measurement is used for attenuation correction.

### Example 7

### Determination of Cerebral Blood Flow in AD Patients

Cerebral blood flow is assessed in patients having probable/mild AD or in patients having an elevated risk of developing AD (see Example 5) essentially as described by Alsop et al. (2000) Ann. Neurol. 47: 93-100. Arterial spin-labeled blood flow images are acquired in multiple contiguous 5 mm axial sections. Adiabatic electromagnetic labeling is applied at the cervicomedullary junction to invert flowing spins in the carotid and vertebral arteries. Representative radiofrequency irradiation amplitude is 36 mG and a magnetic field gradient is 0.25 gauss/cm. Labeled images are subtracted from control images acquired using amplitude-modulated control irradiation of about 250 Hz or about 125 Hz applied to the same location. Imaging is performed in a 1.5 T MRI scanner. Blood flow images are transformed to a standard anatomical space for voxel-by-voxel analysis.

### Example 8

### Determination of Neurochemical Profile in AD Patients

Neurochemical profile is assessed in patients having probable/mild AD or in patients having an elevated risk of developing AD (see Example 5) using MR imaging in conjunction with spectroscopy, essentially as described by Krishnan et al. (2003) Am. J. Psychiatry 160: 2003-2011. MRI and ¹H-MRS studies are performed using a 1.5 T magnetic source. Axial T1-weighted three-dimensional gradient echo MRI may be acquired with 1.5 mm contiguous slices (matrix=256×128, field of view=22 cm). Imaging at the level of the third ventricle provides cortical gray, periventricular, white, and subcortical gray matter in a single slice. ¹H-MRS (two-dimensional chemical-shift imaging) is used to assess amplitudes and areas of N-acetylaspartate, choline moieties, creatine, myo-inositol, etc. See also Lazeyras et al. (1998) Psychiatry Res. 82: 95-106 and Charles et al. (1996) Magn. Reson. Med. 35: 606-610.

### Example 9

### Determination of Neuronal Integrity in AD Patients

Neuronal integrity is assessed in patients having probable/mild AD or in patients having an elevated risk of developing AD (see Example 5) using iodine-123 quinuclidinyl benzilate (¹²³I-QNB) SPECT imaging of the M1 muscarinic receptor. In brief, the (R,R) QNB isomer is synthesized and labeled with ¹²³I using a high performance liquid chromotographic technique, essentially as described by Weinberger et al. (1992) Clin. Neuropharmacol. 15 Suppl 1 Pt A:194A-195A. The labeled QNB is administered to AD patients by intravenous injection at a dosage of approximately 160 Mq. Tomographic images are acquired using a SMV DST-XL dual head gamma camera. The projections are prefiltered, corrected for decay and attenuation, and reconstructed with a ramp filter. Reconstructed images are registered to a single SPECT template image set in standardized stereotactic space, smoothed, and normalized to the mean count within the image. Group comparisons of the treated and control groups are assessed using a statistical parametric software program such as SPM99. See Kemp et al. (2003) J. Neurol. Neurosurg. Psychiatry 74: 1567-1570.

### Example 10

### Screening Assays to Identify Disease-Modifying AD Therapies

Candidate disease-modifying drugs are tested in AD animal models. Candidate drugs are administered in a variety of amounts and/or combinations and by any suitable route, for example, intravenous injection or direct injection into brain. Following drug administration, disease biomarkers are assessed as described in Examples 24. Therapeutic response is assessed by detecting changes in one or more disease biomarkers at preclinical stages, such that the altered profile of one or more disease biomarkers is more similar to that observed in control animals.

For example, a short-term screening assay is conducted in which Tg2576 or PSAPP mice are treated for 1 week, beginning when initial observations of significant changes in hippocampal function are detected in the above-noted observational study. One or more candidate drugs is administered to Tg2576 or PSAPP mice followed by weekly monitoring of disease biomarkers until their return to control levels (i.e., relevant measures of biomarkers in control animals).

All references cited in the instant specification are incorporated herein in their entirety to the extent that they support and enable practice of the disclosed invention.

1. A method for identification of compounds for the treatment of a neurodegenerative disorder during preclinical stages, the method comprising:
(a) administering one or more candidate compounds to a preclinical animal model of a neurodegenerative disorder;
(b) assessing changes in one or more disease biomarkers in the animal model relative to measures of the one or more disease biomarkers in a control animal; and
(c) selecting a candidate compound that induces a change in one or more disease biomarkers toward measures of the one or more disease biomarkers in a control animal.

2. The method of claim 1, wherein the neurodegenerative disorder is Alzheimer's disease.

3. The method of claim 2, wherein the animal model is a Tg2576 mouse.

4. The method of claim 2, wherein the animal model is a PSAPP mouse.

5. The method of claim 1, wherein the disease biomarker is a neuroimaging biomarker.

6. The method of claim 5, wherein the neuroimaging biomarker is cerebral blood flow.

7. The method of claim 6, wherein cerebral blood flow is assessed by arterial spin labeling (ASL).

8. The method of claim 5, wherein the neuroimaging biomarker is glucose utilization.

9. The method of claim 8, wherein glucose utilization is assessed by [¹⁸F]fluordeoxyglucose (FDG) PET.

10. The method of claim 5, wherein the neuroimaging biomarker is a level of a brain metabolite.

11. The method of claim 10, wherein the level of a brain metabolite is assessed by magnetic resonance spectroscopy (MRS).

12. The method of claim 1, further comprising
(d) assessing changes in two or more neuroimaging, genetic, or molecular disease biomarkers in the animal model relative to measures of the two or more neuroimaging, genetic, or molecular disease biomarkers in a control animal.

13. A method for identification of disease-modifying compounds for the treatment of a neurodegenerative disorder during preclinical stages, the method comprising:
(a) administering one or more candidate compounds to a preclinical animal model of a neurodegenerative disorder;
(b) assessing changes in one or more disease biomarkers in the animal model relative to measures of the one or more disease biomarkers in a control animal; and
(c) selecting a candidate compound that induces a change in one or more disease biomarkers toward measures of the one or more disease biomarkers in a control animal.

14. The method of claim 13, wherein the neurodegenerative disorder is Alzheimer's disease.

15. The method of claim 14, wherein the animal model is a Tg2576 mouse.

16. The method of claim 14, wherein the animal model is a PSAPP mouse.

17. The method of claim 13, wherein the disease biomarker is a neuroimaging biomarker.

18. The method of claim 17, wherein the neuroimaging biomarker is cerebral blood flow.

19. The method of claim 18, wherein cerebral blood flow is assessed by arterial spin labeling (ASL).

20. The method of claim 17, wherein the neuroimaging biomarker is glucose utilization.

21. The method of claim 20, wherein glucose utilization is assessed by [¹⁸F]fluordeoxyglucose (FDG) PET.

22. The method of claim 17, wherein the neuroimaging biomarker is a level of a brain metabolite.

23. The method of claim 22, wherein the level of a brain metabolite is assessed by magnetic resonance spectroscopy (MRS).

24. The method of claim 13, further comprising
(d) assessing changes in two or more neuroimaging, genetic, or molecular disease biomarkers in the animal model relative to measures of the two or more neuroimaging, genetic, or molecular disease biomarkers in a control animal.
